(19) United States
(12) Patent Application Publication
(10) Pub. No.: US 2025/0161185 A1
(43) Pub. Date: May 22, 2025

(54) POLYGLYCOSYLATED RUTIN DERIVATIVES FOR REDUCING FACIAL SKIN REACTIONS

(71) Applicant: GIVAUDAN SA, VERNIER (CH)

(72) Inventors: Carole LAMBERT, Toulouse (FR); Daniel AURIOL, Toulouse (FR); Morgane DE TOLLENAERE, Pomacle (FR); Amandine SCANDOLERA, Pomacle (FR); Romain REYNAUD, Toulouse (FR)

(21) Appl. No.: 18/695,111

(22) PCT Filed: Sep. 26, 2022
(86) PCT No.: PCT/EP2022/076705

§ 371 Date: Mar. 25, 2024

(30) Foreign Application Priority Data
Sep. 27, 2021 (GB) 2113755.9

(51) Int. Cl. A61K 8/60; A61Q 19/00

(52) U.S. Cl. CPC A61K 8/602; A61Q 19/005; A61K 2800/75

(57) ABSTRACT

A method of reducing the deleterious effect on the skin exposed to prolonged face mask wearing, comprising the application to the skin potentially affected, prior to mask wearing, of a preparation comprising a polyglycosylated rutin having the formula

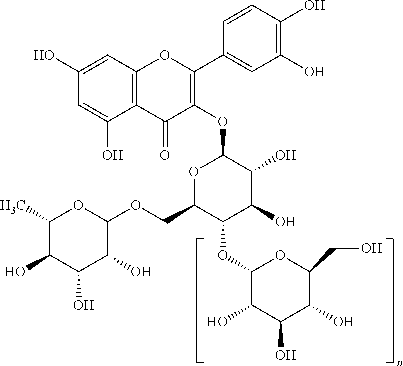

in which over 50% molarity of the glycosylated rutin has n greater than 2.

The known effects of prolonged mask wearing (red spots, vascularisation, hyperpigmentation) are considerably reduced and even completely prevented.

This disclosure relates to a method of providing protection to skin that is subject to extended mask-wearing.

“Maskne” is a phenomenon regularly occurring people who are required to wear masks for prolonged periods, particularly in the medical professions, or for respiratory protection for workers exposed to fine dusts. When a mask wearer talks or breathes, the mask tends to trap hot air and breathed-out water vapour. This creates a warm, humid environment, ideal for the growth of bacteria and other flora, such as fungal species, plus types of skin mites. This can lead to acne, rosacea visible pigmentary changes, as well as perioral dermatitis and inflammatory oedema, thus giving both unsightly skin conditions and discomfort and even pain.

The recent SARS-Cov-2 virus pandemic with the spread of prolonged-period mask-wearing to the general public, for example, in stores or on journeys in public transport has meant that these problems are now much more widespread. A substantial proportion of the population finds this uncomfortable, not only for the stuffiness feeling that this induces, particularly in hot weather, but also for the skin problems mentioned previously, especially in the case of masks that are not properly fitted.

It has now been found that it is possible to reduce considerably, or even completely, the problems associated with long-duration face mask-wearing. There is therefore provided a method of reducing the deleterious effect on the skin exposed to prolonged face mask wearing, comprising the application to the skin potentially affected, prior to mask wearing, of a preparation comprising a polyglycosylated rutin having the formula

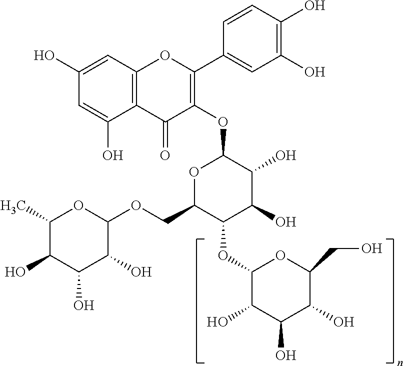

in which over 50% molarity of the glycosylated rutin has n greater than 2.

There is additionally provided the use of a polyglycosylated rutin as hereinabove defined in the preparation of a product for application to the skin for the prevention of deleterious effects of prolonged mask wearing.

The glycosylation of rutin, a polyphenolic flavonoid, is well known in the art, for example, from Journal of Microbiology and Biotechnology Volume 26 Issue 11, pp. 1845-1854 (2016) and U.S. Pat. No. 5,145,781. Glycosylated rutin is known for its water solubility, superior to that of rutin, thus allowing rutin to be made readily available. It is known to have useful physiological properties and also anti-ageing properties. However, it was not known that it would function as a solution to long-term mask wearing problems.

The glycosylation of rutin takes place on the fourth carbon of the glucose unit. This process invariably results in a spread of values of n, ranging from 1 upwards. In this disclosure, glycosylated rutins where n>2 comprise the majority of glycosylated rutins, higher than 50%, more particularly higher than 60% molarity. In a particular embodiment, the average value of n is from 2-11, particularly at least 3, more particularly from 3-4.

The abovementioned enhancement of water solubility is a noteworthy advantage of the polyglycosylated rutins hereinabove described, especially when n is 3-4. Rutin itself is relatively insoluble (0.125 g/L), but with n=3-4, the water solubility rises to about 1830 g/L. i.e 14,000 times the solubility of rutin. This allows the preparation of a product considerably enriched in rutin.

As mentioned hereinabove, the glycosylation of rutin is well known, and it may be prepared any method known to the art.

The polyglycosylated rutin may be incorporated into a product to be applied to the skin, for example, a cream of a lotion. The proportion of polyglycosylated rutin in such a product may vary widely, depending on the nature of the product, but a typical proportion is from 0.5% to 4%, particularly from 1-2% by weight of the composition.

There is therefore also provided a composition adapted to be applied to the skin for the prevention of deleterious effects of prolonged mask-wearing, the composition comprising a polyglycosylated rutin as hereinabove described.

Such products may contain the usual ingredients used in facially-applied products, in art-recognised proportions. Typical, non-limiting ingredients include water, chelating agents, UV absorbers, moisturising agents, preservatives, thickening agents, silicones, essential oils, fragrances, rheology modifiers, vitamins, pharmaceutical ingredients, antioxidants, humectants and emulsifiers

It has been found that the application of such products to the skin prior to an extended period of mask wearing substantially reduces the deleterious effects commonly experienced in such situations.

The disclosure is further described with reference to the following examples, which describe particular embodiments, and which are not meant to be in any way limiting.

### EXAMPLE 1

### Preparation of Polyglycosilated Rutin.

The Following Ingredients were Used:
Reaction medium composition:
   Rutin 131.5 g
   Alpha-cyclodextrin 658 g
   Sodium acetate buffer pH 5.7 4500 g
   cyclodextrin-glucanotransferase* 6.57 g (22.3kNU)
      *Toruzyme™ 3.0 L was used.

The ingredients were blended and stirred at 70° C. for 16 hours. The reaction was stopped by the addition of sulphuric acid of pH no less than 2.5 and the mixture was filtered. The filtrate was purified on adsorbent resin to remove undesirable by-products and rutin derivatives were recovered by eluting the resin with a mixture of ethanol and water (>60%).

Analysis of the product showed the following results—G1=1 glucoside unit, G2=2 glucoside units, G3=3 glucoside units, etc.

### G1

¹H NMR (DMSO-d6): 0.95, 3.06, 3.11, 3.24, 3.26, 3.29, 3.31, 3.34, 3.37, 3.4, 3.42, 3.43, 3.43, 3.48 3.49, 3.57, 3.74, 4.44, 4.98, 5.37, 6.2, 6.39, 6.86, 7.53, 7.53 ppm.

¹³C NMR (DMSO-d6): δ=18.1, 60.9, 67.3, 68.7, 69.9, 70.8, 70.9, 72.2, 72.8, 73.6, 73.9, 74.4, 76.6, 80.5, 94, 99.1, 101.2, 101.4, 101.7, 104.3, 115.6, 116.6, 121.5, 122, 133.6, 145.1, 148.8, 156.7, 157.1, 161.6, 164.4, 177.7 ppm. MS: m/z (%) 609 (100) [M⁺].

### G2

¹H NMR (DMSO-d6): 0.95, 3.06, 3.07, 3.23, 3.26, 3.3, 3.3, 3.3, 3.36, 3.36, 3.37, 3.42, 3.42, 3.45, 3.51, 3.53, 3.61, 3.63, 4.42, 4.99, 4.99, 5.38, 6.19, 6.39, 6.85, 7.53, 7.53 ppm.

¹³C NMR (DMSO-d6): δ=18.1, 60.4, 61.1, 66.8, 68.7, 70.2, 70.8, 70.9, 72.2, 72.2, 72.3, 73, 73.5, 73.6, 73.9, 74, 74.3, 76.4, 79.7, 80.6, 94.1, 99.1, 101, 101.3, 101.4, 101.4, 104.2, 115.7, 116.6, 121.4, 121.9, 133.5, 145.1, 148.9, 156.8, 157, 161.3, 164.7, 177.6 ppm. MS: m/z (%) 934 (100) [M⁺].

### G3

¹H NMR (DMSO-d6): 0.95, 3.06, 3.07, 3.22, 3.26, 3.3, 3.3, 3.3, 3.3, 3.37, 3.37, 3.37, 3.38, 3.42, 3.42, 3.47, 3.5, 3.55, 3.55, 3.6, 3.63, 3.63, 4.42, 4.98, 4.99, 5, 5.37, 6.18, 6.37, 6.85, 7.52, 7.54.

¹³C NMR (DMSO-d6): δ=18.1, 60.4, 61.1, 66.8, 68.6, 70.2, 70.8, 70.9, 72.1, 72.1, 72.2, 72.3, 72.3, 72.9, 73.4, 73.4, 73.6, 73.8, 74, 74.3, 76.3, 79.7, 79.8, 80.6, 94, 99.2, 101, 101, 101.4, 101.4, 104.1, 115.7, 116.5, 121.4, 121.9, 133.5, 145.1, 148.9, 156.8, 156.9, 161.5, 165, 177.6 ppm. MS: m/z (%) 934 (100) [M⁺].

### G4 MS: m/z (%) 1259 (100) [M⁺].

### G5 MS: m/z (%) 1421 (100) [M⁺].

### G6 MS: m/z (%) 1583 (100) [M⁺].

### G7 MS: m/z (%) 1745 (100) [M⁺].

### G8 MS: m/z (%) 1907 (100) [M⁺].

### G9 MS: m/z (%) 1035 (100) [M⁺].

### G10 MS: m/z (%) 1116 (100) [M⁺].

### G11 MS: m/z (%) 1197 (100) [M⁺].

The average value of n in the formula above was 4.

### EXAMPLE 2

### Formulation of a Skin Cream.

A skin cream was prepared by mixing the following ingredients (all parts by weight):

| | |
|---|---|
| WATER | 87.30 |
| EMULIUM ™ DELTA MD* | 5.0 |
| ISODECYL NEOPENTANOATE | 4.5 |
| PRODUCT OF EXAMPLE 1 | 1.0 |
| GLYCERIN | 1.0 |
| PHENOXYETHANOL | 0.5 |
| DIMETHICONE | 0.3 |
| 1.2 HEXANEDIOL, CAPRYLYL | 0.3 |
| GLYCOL | |
| FRAGRANCE | 0.1 |

*A commercially-available emulsifier (ex Gattefossé)

This cream is hereinafter designated as “Example”

For purposes of comparison, a placebo skin cream was prepared according to the formula shown above, but replacing the product of Example 1 with one further part of water. This cream is hereinafter designated as “placebo”

### EXAMPLE 3

### Testing of the Creams

The subjects were 20 women, aged at least 18 and known to have sensitive skin around the mouth.

The subjects applied the creams to the face twice daily, once in the morning and once in the evening. They applied both creams, one to each half of the face, consistently using the same cream on each side of the face, without knowing the identities of the creams. They wore surgical masks for 2 hours.

### (a) Red Spot and Vascularisation Testing.

One of the problems of prolonged mask wearing is the occurrence of small red spots on the skin. Another is vascularization, the increase in redness in the face as a result of the growth of capillaries near the surface of the skin.

Spots can be assessed by simply counting the number. Vascularization can be assessed using a polarized light from VISA® 2.3.

Digital photographs of the face were made at different times with Visia® CR 2.3 from Canfield® imaging systems. The control of the repositioning takes place directly on data-processing screen using an overlay visualization of the images at each time of acquisition. The Visia® CR 2.3 allows taking pictures with different types of illuminations and a very rapid capture of images. A series of photos were taken under multi-spectral imaging and analysis allow capturing visual information affecting appearance of the skin. The photos were made on each hemi-face.

The analyses were done using Maestro® software on RBX pictures for red spots and features (vascularization) measurements. The RBX pictures highlight red spots and skin vascularization, and the software quantifies the number of red sports and red features (meaning red spot+vascularization). Soothing efficacy is demonstrated by a reduction in red spots and vascularisation.

After 24 hours, it was found that, in areas treated with the placebo, the number of red spots increased by an average of 15.3%, whereas, in areas treated with the Example, the number of spots decreased by 23.1%, meaning that the total red spots difference over 24 hours was 38.4%.

An assessment of overall red features (spots+vascularization) showed an overall reduction of 6.1% (4.5% increase for the placebo, 1.6% decrease for the Example).

### (b) Hyperpigmentation Test

Another common problem with prolonged mask-wearing is hyperpigmentation, which is the appearance of brown spots on the mask area. These were assessed by counting after 24 hours and after 4 days. This assessment was carried out using the methods, Visia®CR 2.3 equipment and Maestro® software described above.

The analyses were done using on standardized pictures for brown spots measurement.

After 24 hours, the subjects showed an overall improvement of 3.4% (1.3% more brown spots for the placebo, 2.1% fewer for the Example), and after 4 days, the overall improvement was 3.5% (1.1% more for the placebo, 2.4% fewer for the Example).

### Subject Self-Assessment

The subjects were asked to assess their individual creams after 24 hours. The results are shown below:

| | Example | Placebo |
|---|---|---|
| Did you skin feel more comfortable after | 74% | 42% |
| treatment? | | |
| Did your skin appear softer after treatment? | 74% | 53% |
| Was you skin less tight after treatment? | 74% | 53% |
| Was your skin more hydrated after | 68% | 47% |
| treatment? | | |

The invention claimed is:

1. A method of reducing the deleterious effect on the skin exposed to prolonged face mask wearing, comprising the application to the skin potentially affected, prior to mask wearing, of a preparation comprising a polyglycosylated rutin having the formula 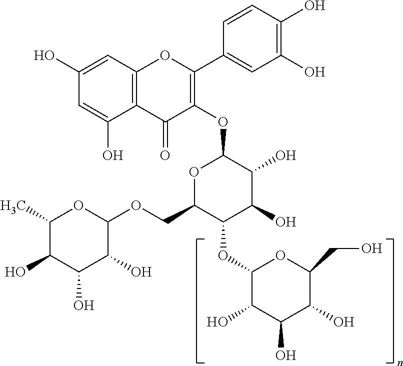
in which over 50% molarity of the glycosylated rutin has n greater than 2.

2. The method according to claim 1, in which over 60% molarity of the glycosylated rutin has n of at least 2.

3. The method according to claim 1, in which n is from 2-11.

4. A method of utilizing a polyglycosylated rutin of the formula 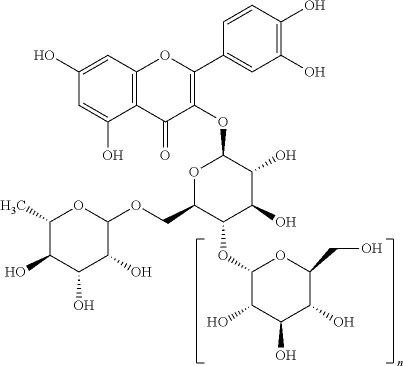
in which over 50% molarity of the glycosylated rutin has n greater than 2 in the preparation of a product for application to the skin for the prevention of deleterious effects of prolonged mask wearing.

5. The method according to claim 4, in which n is from 2-11.

6. A composition configured to be applied to the skin for the prevention of deleterious effects of prolonged mask-wearing, the composition comprising a polyglycosylated rutin of the formula 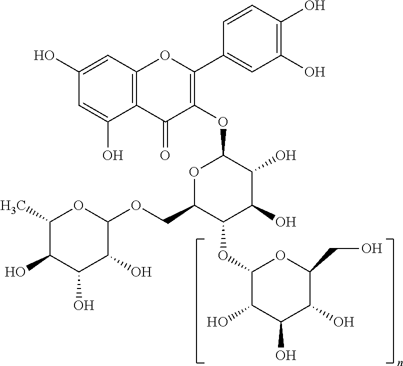
in which over 50% molarity of the glycosylated rutin has n greater than 2.

7. The composition according to claim 6, in which n is from 2-11.

8. The method according to claim 3, in which n is at least 3.

9. The method according to claim 8, in which n is from 3-4.

10. The method according to claim 5, in which n is at least 3.

11. The method according to claim 10, in which n is from 3-4.

12. The method according to claim 7, in which n is at least 3.

13. The method according to claim 12, in which n is from 3-4.
